(19) United States
(12) Patent Application Publication
(10) Pub. No.: US 2009/0187168 A1
(43) Pub. Date: Jul. 23, 2009

(54) SLIDE MEMBER ADVANCING/RETRACTING MECHANISM

(75) Inventor: Masanori MAEDA, Tokyo (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/349,600

(22) Filed: Jan. 7, 2009

(30) Foreign Application Priority Data
Jan. 18, 2008 (JP) 2008-8634

(51) Int. Cl. A61B 17/94; G05G 1/00; F16H 35/00

(52) U.S. Cl. 606/1; 74/503; 74/842

(57) ABSTRACT

A slide member advancing/retracting mechanism includes an outer sheath; a slide member installed in the outer sheath to be slidable in an axial direction without rotating relative to the outer sheath; and an operating portion which can be freely rotated relative to, and about a common axis of, the outer sheath and the slide member. The operating portion can be operated so as to linearly advance/retract the slide member within the outer sheath.

### BACKGROUND OF THE INVENTION

1. Field of the Invention

The present invention relates to a mechanism for advancing and retracting a slide member which is inserted into an outer sheath and supported thereby without varying the rotational position (rotational angle) of the slide member relative to the outer sheath.

2. Description of the Prior Art

For instance, in the field of medical equipment, especially the field of endoscopic treatment tools, a mechanism including an outer sheath and a slide member which is slidably inserted into the outer sheath, wherein the outer sheath is provided at the rear end thereof with a finger hook, the slide member is provided at the rear end thereof with a force-applied portion (finger insertion hole) and the slide member advances and retracts by applying a relative force in the axial direction of the slide member between the finger hook and the force-applied portion, is known in the art.

However, this mechanism requires a lot of skill in adjusting the axial force applied between the finger hook and the force-applied portion, and it is extremely difficult to delicately adjust the length of projection of the slide member from the distal end of the outer sheath. In other words, since the direction of the force applied to the slide member (operating direction) to advance and retract the slide member is coincident with the axis of the whole device, there is a possibility of the whole device being accidentally squeezed into or pulled out of the body (patient). In endoscopic operations with a limited view, accidental displacements of the distal end of a treatment tool are extremely dangerous. Namely, this conventional structure has a critical problem (critical defect) with the safety and operability thereof.

In addition, in the endoscopic treatment tool which has been under development by the assignee of the present invention, investigation into limiting relative rotation between the outer sheath and the slide member and controlling the rotational angle of the slide member caused by the rotational angle of the outer sheath has been carried out. However, in this sort of mechanism in which relative rotation between the outer sheath and the slide member is limited, if an advancing/retracting mechanism in which the outer sheath is provided at the rear end thereof with a finger hook and in which the slide member is provided at the rear end thereof with a force-applied portion (finger insertion hole) is adopted, the direction of the finger insertion hole (operating portion) at the rear end of the slide member is determined by the rotational angle of the outer sheath (the slide member), which singularly deteriorates the operability of the endoscopic treatment tool. Namely, depending on the rotational angle of the outer sheath (the slide member), a state where a finger cannot be inserted into the finger insertion hole arises.

### SUMMARY OF THE INVENTION

The present invention has been devised based on an awareness of the issues described above and provides a mechanism for advancing and retracting a slide member which is inserted into an outer sheath in a manner to prevent the slide member from rotating relative to the outer sheath, wherein the mechanism is structured to allow the orientation of the operating portion to be freely changed relative to the outer sheath.

In addition, the present invention provides a slide member advancing and retracting mechanism which is structured to make it possible to make a fine adjustment to the amount (length) of projection of the slide member from the outer sheath in an easy manner.

According to an aspect of the present invention, a slide member advancing/retracting mechanism is provided, including an outer sheath; a slide member installed in the outer sheath to be slidable in an axial direction without rotating relative to the outer sheath; and an operating portion which can be freely rotated relative to, and about a common axis of, the outer sheath and the slide member, wherein the operating portion can be operated so as to linearly advance/retract the slide member within the outer sheath.

It is desirable for the operating portion to include an operating wheel holder rotatable about the common axis of the outer sheath and the slide member; and an operating wheel supported by the operating wheel holder to be rotatable on a rotational shaft, an axis of which extends orthogonally to the common axis without intersecting therewith. The operating wheel and the slide member are engaged with each other so that the slide member linearly moves forward and backward by forward and reverse rotations of the operating wheel, respectively.

It is desirable for the slide member to include an axisymmetrical rack formed integrally with the slide member and having rack teeth in a rotationally-symmetrical shape about the common axis. It is desirable for the operating portion to include a pinion holder supported by the outer sheath to be rotatable about the common axis; and a pinion which is supported by the pinion holder to be freely and manually rotatable, and remains in mesh with the axisymmetrical rack regardless of a rotational position of the pinion holder relative to the outer sheath about the common axis.

It is desirable for the slide member to include a relatively-rotatable slide member which is connected to a rear end of the slide member to be freely rotatable relative to the slide member and to move with the slide member in the axial direction. It is desirable for the operating portion to include a pinion holder supported by the outer sheath to be rotatable about the common axis; and a pinion which is supported by the pinion holder to be freely and manually rotatable, and meshes with a rack formed on the relatively-rotatable slide member.

It is desirable for the outer sheath to be formed to serve as an endoscopic treatment tool which is inserted into a human body, and for the slide member to include a support member, provided at a distal end of the slide member, for supporting at least one of a tool and a treatment material which is used for performing a treatment in the human body.

It is desirable for the operating portion to be substantially coaxial with the outer sheath.

It is desirable for the rotational shaft to be integral with the operating wheel holder.

It is desirable for the operating wheel holder to include a pair of half holders which holds the pinion therebetween.

It is desirable for the operating portion to be fixed at a rear end of the outer sheath.

It is desirable for the slide member advancing/retracting mechanism to be incorporated in an endoscopic treatment tool.

In an embodiment, a slide member advancing/retracting mechanism is provided, including an outer sheath, a slide member installed in the outer sheath to be slidable in an axial direction thereof without rotating relative to the outer sheath, an operating portion freely rotatable relative to the outer sheath about the axis of the outer sheath, and a rack and pinion mechanism installed between the operating portion and the slide member. Manually rotating a pinion of the rack and pinion mechanism causes the slide member to advance and retract in the axial direction via the rack and pinion mechanism.

More generally, the slide member advancing/retracting mechanism according to the prevent invention can be widely used as an operating mechanism for performing some sort of operation not only in a human body but also in a small, narrow space.

Due to slide member advancing/retracting mechanism including an outer sheath, a slide member installed in the outer sheath to be slidable in an axial direction without rotating relative to the outer sheath, and an operating portion which can be freely rotated relative to, and about a common axis of, the outer sheath and the slide member, wherein the operating portion can be operated so as to linearly advance/retract the slide member within the outer sheath, the slide member can be operated to advance and retract with the rotational angle of the operating portion, about the common axis of the outer sheath and the slide member, being freely changed. In addition, the amount (length) of projection of the slide member from the outer sheath can be precisely controlled by adopting a rack and pinion mechanism as the operating portion.

The present disclosure relates to subject matter contained in Japanese Patent Application No. 2008-8634 (filed on Jan. 18, 2008) which is expressly incorporated herein by reference in its entirety.

### BRIEF DESCRIPTION OF THE DRAWINGS

The present invention will be described below in detail with reference to the accompanying drawings in which:

FIG. 1 is a plan view of an embodiment of a slide member advancing/retracting mechanism according to the present invention;

FIG. 2 is a front view of the slide member advancing/retracting mechanism shown in FIG. 1;

FIG. 3 is a cross sectional view taken along the III-III line shown in FIG. 1;

FIG. 4 is a cross sectional view taken along the IV-IV line shown in FIG. 2, viewed in the direction of the appended arrows;

FIG. 5 is an exploded perspective view of a main part of the slide member advancing/retracting mechanism shown in FIG. 1;

FIG. 6 show diagrams showing a procedure for coupling a pair of half pinion-holders and a front coupling ring (or a rear coupling ring) to each other in the slide member advancing/retracting mechanism shown in FIGS. 1 through 5;

FIG. 7 is a longitudinal cross sectional view of another embodiment of the slide member advancing/retracting mechanism according to the present invention;

FIG. 8 is an exploded perspective view of a part of the slide member advancing/retracting mechanism shown in FIG. 7, showing the joint between a slide member and a relatively-rotatable slide member;

FIG. 9 is a cross sectional view taken along the IX-IX line shown in FIG. 7;

FIG. 10 is a perspective view of a part of a therapeutic-substance carrying/administering appliance (to which an embodiment of a slide member advancing/retracting mechanism according to the present invention is applied) at the distal end of the outer sheath thereof, showing a part of the outer sheath in cross section, in a state where a sheet supporting element projects outward from the distal end of the outer sheath and has fully expanded, and a sheet-shaped therapeutic-substance which is to be carried by the therapeutic-substance carrying/administering appliance;

FIG. 11 is a longitudinal cross sectional view of a part of the therapeutic-substance carrying/administering appliance in the vicinity of the junction between the slide member and a support member;

FIG. 12 is an exploded perspective view of a part of the therapeutic-substance carrying/administering appliance in the vicinity of the distal end of the outer sheath thereof, showing a state where a combination of the support member and the sheet supporting element is removed from the distal end of the slide member;

FIG. 13 is an exploded perspective view of the support member and the sheet supporting element;

FIG. 14 is a perspective view of a portion of the therapeutic-substance carrying/administering appliance in the vicinity of the distal end of the outer sheath thereof and a sheet-shaped therapeutic-substance which is to be carried by the therapeutic-substance carrying/administering appliance, showing a state where the sheet supporting element projects from the distal end of the outer sheath and has fully expanded into a flat shape;

FIG. 15 is a perspective view of the portion of the therapeutic-substance carrying/administering appliance shown in FIG. 14, wherein the sliding member has been slightly retracted into the distal end of the outer sheath from the state shown in FIG. 14, showing a state shortly after the commencement of resilient deformation of the sheet supporting element from the expanded state shown in FIG. 14 to a fully rolled state shown in FIG. 18;

FIG. 16 is a perspective view of the portion of the therapeutic-substance carrying/administering appliance shown in FIG. 14, wherein the sliding member has been slightly retracted further into the distal end of the outer sheath from the state shown in FIG. 15, showing a state where the sheet supporting element has been further deformed from the state shown in FIG. 15 to become closer to the fully rolled state shown in FIG. 18;

FIG. 17 is a perspective view of the portion of the therapeutic-substance carrying/administering appliance shown in FIG. 14, wherein the sliding member has been slightly retracted further into the distal end of the outer sheath from the state shown in FIG. 16, showing a state where the sheet supporting element has been further deformed from the state shown in FIG. 16 to become closer to the fully rolled state shown in FIG. 18; and

FIG. 18 is a perspective view of the portion of the therapeutic-substance carrying/administering appliance shown in FIG. 14, wherein the sliding member has been fully retracted into the distal end of the outer sheath, showing a state where the sheet supporting element has been rolled into a tubular shape and fully accommodated in the outer sheath.

### DESCRIPTION OF THE PREFERRED EMBODIMENT

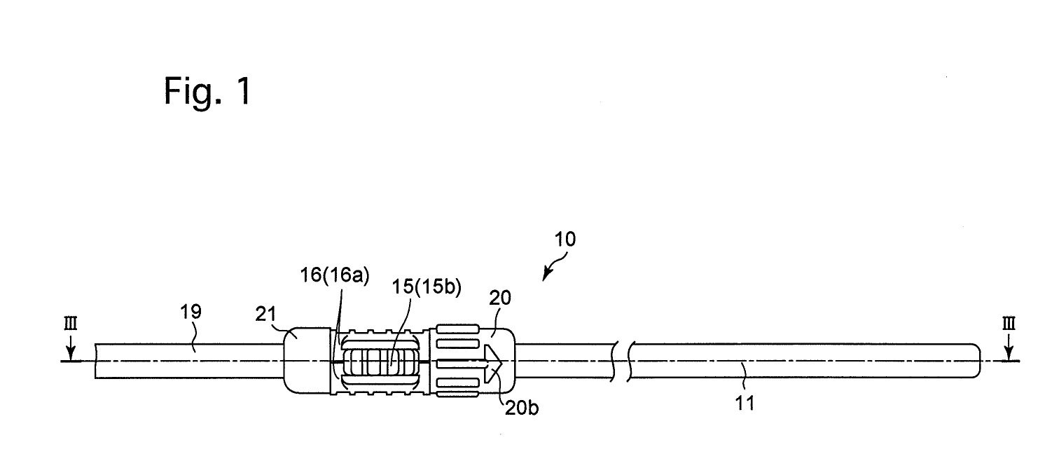

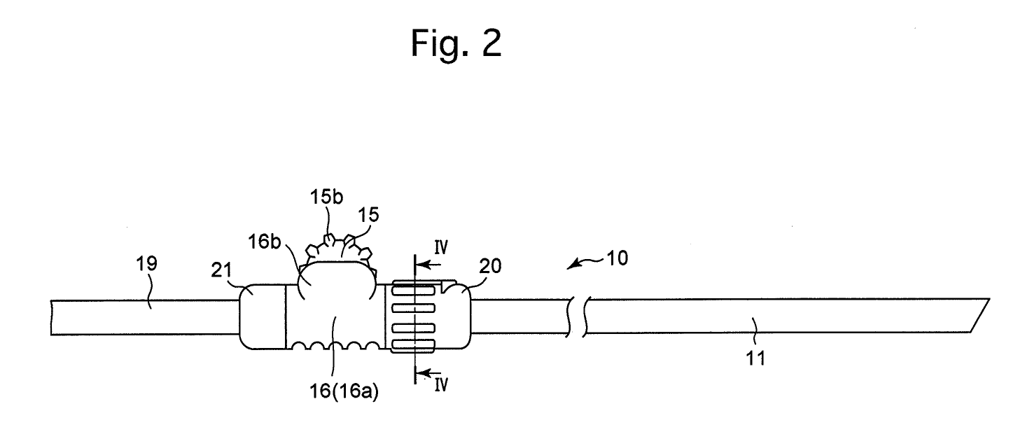

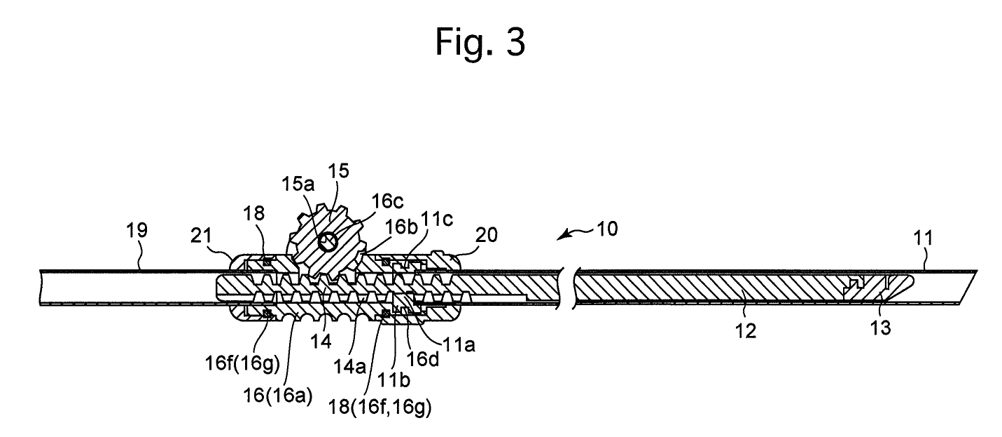

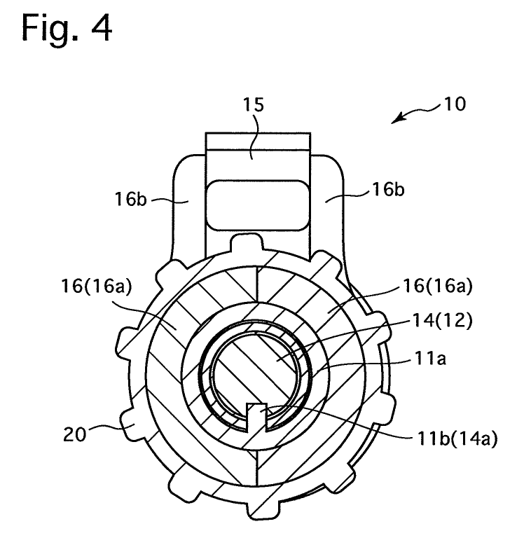

FIGS. 1 through 6 show a first embodiment of a slide member advancing/retracting mechanism 10 according to the present invention. The slide member advancing/retracting mechanism 10 is provided with an outer sheath (endoscopic treatment tool) 11 which is circular in cross section, and is further provided with a slide member (shaft member) 12 which is inserted into the outer sheath 11 to be freely movable in the axial direction thereof. The end surface of the distal end (right end with respect to FIGS. 2 and 3) of the outer sheath 11 is formed as a beveled surface which is inclined to a direction orthogonal to the axial direction of the outer sheath 11 in this particular embodiment. In addition, in the illustrated embodiment, the slide member 12 is provided at the distal end thereof with a separately-formed support member 13. The support member 13 does not rotate relative to the slide member 12, moves with the slide member 12 in the axial direction thereof, and is provided as a part of the slide member 12.

The slide member 12 is provided on a portion thereof in the vicinity of the rear end of the slide member 12 with an axisymmetrical rack 14. The axisymmetrical rack 14 is made by forming rack teeth in a rotationally-symmetrical shape about the axis of the slide member 12. The slide member 12 is provided with a key groove 14a which is formed to extend parallel to the axis of the slide member 12 in a manner to remove part of the axisymmetrical rack 14.

On the other hand, the outer sheath 11 is provided, on an outer peripheral surface thereof at the rear end of the outer sheath 11, with an end ring 11a which is integrally joined to the major part of the outer sheath 11 (to be provided as part of the outer sheath 11). The end ring 11a is provided on an inner peripheral surface thereof with a key projection 11b which projects radially inwards to be engaged in the key groove 14a. The end ring 11a can be molded integrally with the outer sheath 11. The axisymmetrical rack 14 of the slide member 12 projects (can be made to project) from the end ring 11a (the rear end of the outer sheath 11) in the axial direction of the slide member 12.

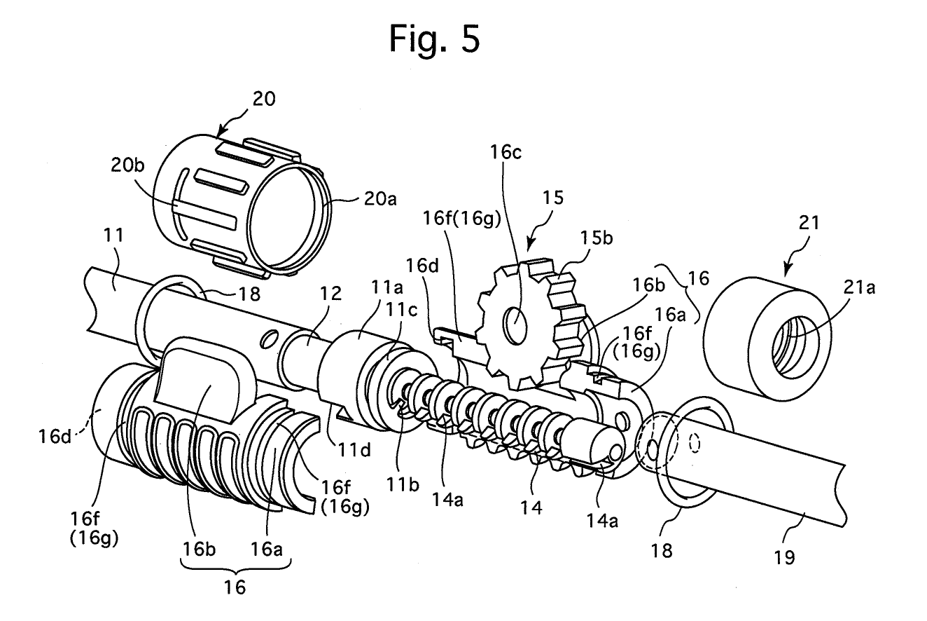

The slide member advancing/retracting mechanism 10 is provided with a pinion (operating wheel) 15 which meshes with the axisymmetrical rack 14 of the slide member 12 in the vicinity of the proximal end of the outer sheath 11. The pinion 15 is supported between a pair of half pinion-holders (operating wheel holder) 16 to be freely rotatable. Each of the pair of half pinion-holders (half holders) 16 is provided with a half-cylindrical part 16a and a pinion support arm 16b which projects in a radial direction from the half-cylindrical part 16a. The pinion support arm 16b of one of the pair of half pinion-holders 16 is provided with a shaft projection (rotational shaft) 16c (see FIGS. 3 and 5) which is inserted into a shaft hole 15a of the pinion 15. The axis of the shaft hole 15a (the axis of the shaft projection 16c) extends orthogonally to the axis of the outer sheath 11 (which is coincident with the axis of the slide member 12) without intersecting therewith. Additionally, the pair of half pinion-holders 16 is provided, at the ends thereof on the end ring 11a side, with a pair of half inner flanges 16d (FIGS. 3 and 5), respectively, which are formed to be engaged in an annular groove 11c formed on the end ring la of the outer sheath 11. Combining the pair of half pinion-holders 16 so as to bring the pair of half inner flanges 16d into engagement in the annular groove 11c, and so as to position the pinion 15 between the pinion support arms 16b with the shaft projection 16c being inserted in the shaft hole 15a of the pinion 15, causes teeth 15b of the pinion 15 to mesh with the axisymmetrical rack 14, and also makes it possible for the combined pair of half pinion-holders (pinion holder) 16 to rotate about the axis of the outer sheath 11 (the axis of the slide member 12). The teeth 15b of the pinion 15 remain in mesh with the axisymmetrical rack 14 regardless of the rotational position of the pair of half pinion-holders 16 about the axis of the slide member 12.

The pair of half pinion-holders 16 is provided, on outer peripheral surfaces thereof in the close vicinity of each of the front and rear ends of the pair of half pinion-holders 16, with a pair of half round grooves 16g, respectively, which generally form an annular O-ring groove 16f. The pair of half pinion-holders 16 is temporarily joined together by fitting two O-rings 18 in the front and rear O-ring grooves 16f, respectively, in a state where the pair of half pinion-holders 16 is put together. Furthermore, a rear protective pipe 19 is held between the rear ends of the pair of half pinion-holders 16 to be fixed thereto. The axisymmetrical rack 14 of the slide member 12 moves forward and rearward inside the rear protective pipe 19 in the axial direction thereof when the slide member 12 advances and retracts relative to the outer sheath 11.

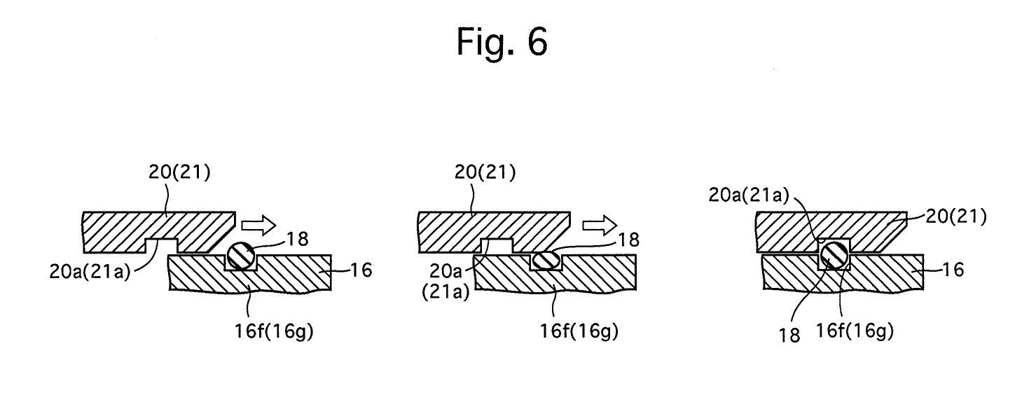

The slide member advancing/retracting mechanism 10 is provided with a front binding ring 20 and a rear binding ring 21 immediately in front of, and behind, the pair of half round grooves 16g, respectively. The front binding ring 20 and the rear biding ring 21 are for permanently joining the pair of half pinion-holders 16 together, which holds the outer sheath 11 and the rear protective pipe 19 at the front and the rear of the pair of half pinion-holders 16, respectively. Namely, the front binding ring 20 and the rear binding ring 21 are fitted onto outer peripheral surfaces of the outer sheath 11 and the rear protective pipe 19 and are slidingly moved to the pair of half pinion-holders 16 in a state where the pair of half pinion-holders 16 is temporarily joined together by the two O-rings 18 with the outer sheath 11 and the rear protective pipe 19 being held between the pair of half pinion-holders 16. The front binding ring 20 and the rear biding ring 21 are provided on inner peripheral surfaces thereof with two annular grooves 20a and 21a which correspond to the O-ring groove 16f (the pair of half round grooves 16g) of the pair of half pinion-holders 16 and the two O-rings 18, respectively. Hence, upon the axially opposed ends of the front and rear binding rings 20 and 21 slidingly moving onto respective front and rear ends of the pair of half pinion-holders 16 and thereafter slidingly moving over the front and rear O-rings 18, respectively, each O-ring 18 is temporarily compressed radially inwards, and subsequently returns to the original shape thereof upon the position of the associated O-ring groove 16f (the pair of half round grooves 16g) coinciding with the position of the associated annular groove 20a or 21a in the axial direction (horizontal direction with respect to FIG. 6) as shown in FIG. 6, which completes the operation for permanently joining the pair of half pinion-holders 16 together. Although neither the front binding ring 20 nor the rear biding ring 21 comes off the pair of half pinion-holders 16 during normal use upon completion of the permanently joining operation, the pair of half pinion-holders 16 can be disassembled by a reverse operation to the above-described permanently joining operation. The front binding ring 20 and the end ring 11a are provided with an anti-rotation projection (not shown) and an anti-rotation groove 11d (see FIG. 5), respectively, which engage with each other in a state where the front binding ring 20 is fitted on the end ring 11a. In addition, the front binding ring 20 is provided thereon with an index mark 20b (see FIGS. 1 and 5) which indicates the orientation (rotational angle) of the outer sheath 11.

In the above described slide member advancing/retracting mechanism 10, the pair of half pinion-holders 16 and the pinion 15 constitute an operating portion in which the pinion 15 and the pair of half pinion-holders 16 can freely rotate together relative to, and about the common axis of, the outer sheath 11 and the slide member 12, while this operating portion can be operated so as to linearly advance/retract the slide member 12 within the outer sheath 11. Namely, taking the orientation (rotational angle) of the outer sheath 11 as a reference, the pair of pinion holders 16 can be rotated relative to, and about the axis of, the outer sheath 11 to any given rotational position. Thereupon, rotating the pinion 15 forward or reverse on the shaft projection 16c, at any given rotational position of the operating portion (which includes the pinion 15 and the pair of half pinion-holders 16) about the common axis of the outer sheath 11 and the slide member 12, causes the slide member 12, which is guided linearly inside the outer sheath 11 by the engagement between the key projection 11b and the key groove 14a, to advance and retract linearly via the engagement of the teeth 15b of the pinion 15 with the rack teeth of the axisymmetrical rack 14.

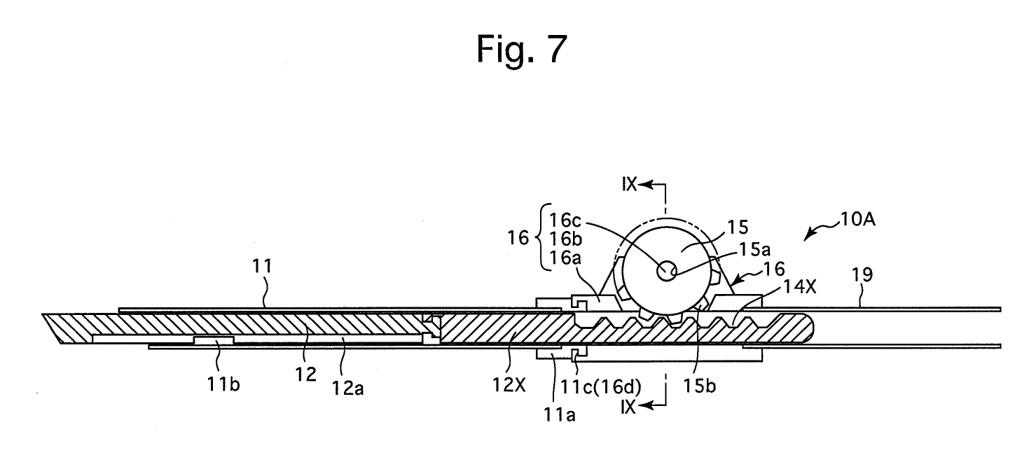

FIGS. 7 through 9 show a second embodiment of the slide member advancing/retracting mechanism 10A according to the present invention. Elements of this mechanism which correspond to those of the first embodiment of the slide member advancing/retracting mechanism 10 are designated by the same reference numerals. In the second embodiment of the slide member advancing/retracting mechanism 10A, a key groove 12a is formed on the slide member 12 that is inserted into the outer sheath 11, and a key projection 11b which is engaged in the key groove 12a is formed on an inner peripheral surface of the outer sheath 11.

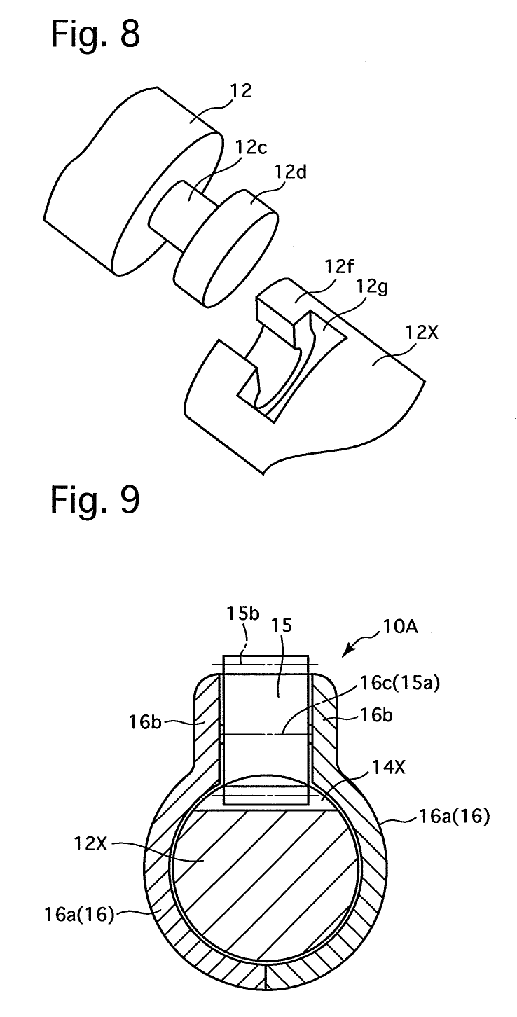

The slide member advancing/retracting mechanism 10A is provided with a relatively-rotatable slide member 12X which is connected to the rear end of the slide member 12 to be freely rotatable relative to the slide member 12 and to move with the slide member 12 in the axial direction thereof. As shown in FIG. 8, this connecting structure is configured so that a small-diameter shaft portion 12c and a large-diameter disk portion 12d are formed in that order (in a rearward direction) at the rear end of the slide member 12 and that a small-diameter groove 12f and a large-diameter groove 12d, in which the small-diameter shaft portion 12c and the large-diameter disk portion 12d are respectively inserted, are formed at the front end of the relatively-rotatable slide member 12X in a manner to prevent the small-diameter shaft portion 12c and the large-diameter disk portion 12d from moving in the axial direction of the slide member 12 while allowing the small-diameter shaft portion 12c and the large-diameter disk portion 12d to rotate freely about the axis of the slide member 12. The relatively-rotatable slide member 12X is provided on a rear end portion thereof with a rack 14X. The rack 14X is a normal type of rack which is formed linearly on a portion of the periphery of the relatively-rotatable slide member 12X along the axis thereof.

A pair of half pinion-holders 16 which is similar to that of the first embodiment of the slide member advancing/retracting mechanism is supported by the end ring 11a, which is fixed at the rear end of the outer sheath 11, to be freely rotatable about the axis of the slide member 12. Namely, the pair of half inner flanges 16d of the pair of half pinion-holders 16 is engaged in the annular groove 11c, which is formed on the end ring 11a of the outer sheath 11, to be freely rotatable relative to the annular groove 11c. In the second embodiment of the slide member advancing/retracting mechanism, the pair of half pinion-holders 16 is fixed to each other by an adhesive, a screw or the like, and the pinion 15 that is engaged with the rack 14X of the relatively-rotatable slide member 12X is supported by a shaft projection 16c (see FIG. 7), which is inserted into a shaft hole 15a of the pinion 15, between the pair of half pinion-holders 16 to be freely rotatable.

According to the second embodiment of the slide member advancing/retracting mechanism 10A, the relatively-rotatable slide member 12X and the pair of half pinion-holders 16 can rotate relative to the outer sheath 11 and the slide member 12 that do not rotate relative to each other. Namely, the pair of half pinion-holders 16 and the pinion 15 constitute an operating portion in which the pinion 15 and the pair of half pinion-holders 16 can freely rotate together relative to, and about the common axis of, the outer sheath 11 and the slide member 12, while this operating portion can be operated so as to linearly advance/retract the slide member 12 within the outer sheath 11. Taking the orientation (rotational angle) of the outer sheath 11 as a reference, the pair of half pinion-holders 16 can be rotated relative to, and about the axis of, the outer sheath 11 to any given rotational position. Thereupon, rotating the pinion 15 forward or reverse on the shaft projection 16c, at any given rotational position of the operating portion (which includes the pinion 15 and the pair of half pinion-holders 16) about the common axis of the outer sheath 11 and the slide member 12, causes the slide member 12, which is guided linearly inside the outer sheath 11 by the engagement between the key projection 11b and the key groove 12a, to advance or retract linearly via the engagement of the teeth 15b of the pinion 15 with rack teeth of the rack 14X.

In the above described first embodiment of the slide member advancing/retracting mechanism 10, similar effects can be obtained even if the pinion 15 and the axisymmetrical rack 14 are replaced by a friction roller and a cylindrical friction column which is in frictional contact with the friction roller, respectively. Likewise, in the above described second embodiment of the slide member advancing/retracting mechanism 10A, similar effects can be obtained even if the pinion 15 and the rack 14X are replaced by a friction roller and a friction plate (member) which is in frictional contact with the friction roller, respectively.

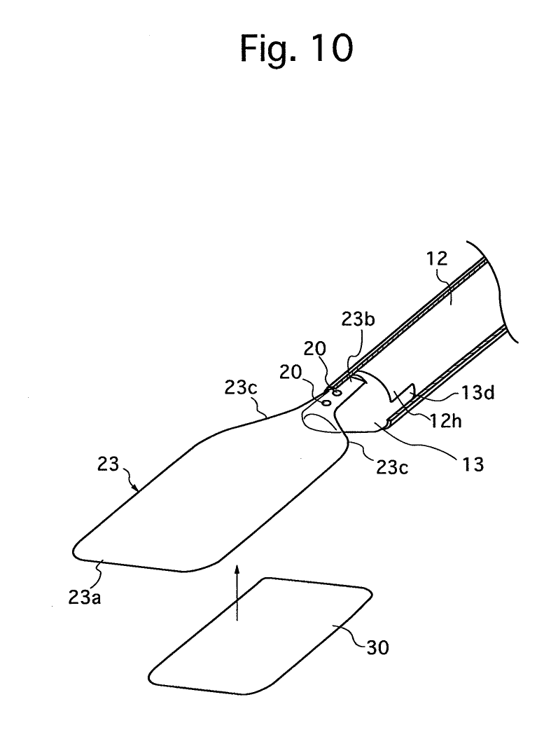

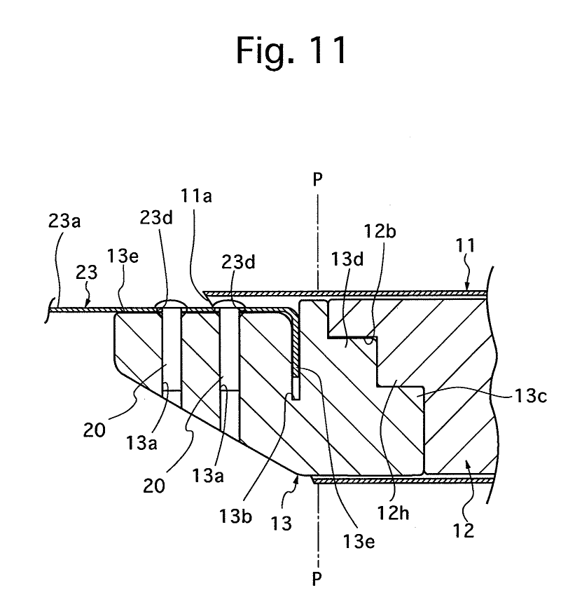

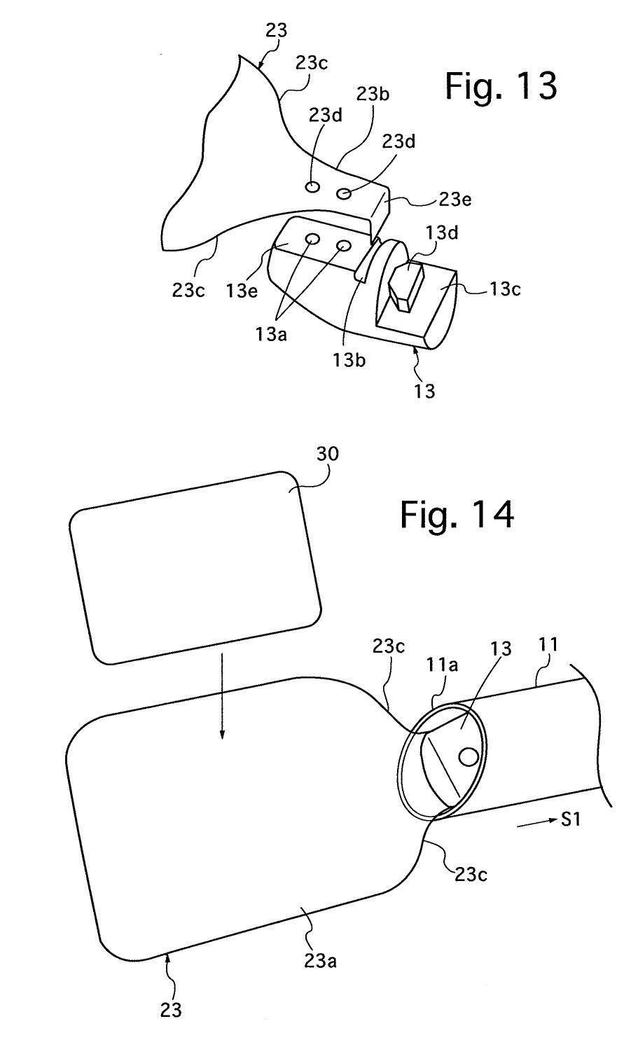

Although the slide member advancing/retracting mechanism according to the present invention can be used for any purpose, a more specific embodiment will be hereinafter discussed with reference to FIGS. 10 through 18. This embodiment is an example of a therapeutic-substance carrying/administering appliance in which a sheet supporting element (tool) 23 for carrying and attaching a sheet-shaped therapeutic substance (treatment material 30 (see FIGS. 10 and 14; hereinafter referred to as a sheet 30) in an endoscopic operation (more specifically, an endoscopic operation which is performed by inserting the outer sheath 11 into a body via a trocar (trockar) previously inserted into the body when a laparoscopic or thoracoscopic operation is performed) is fixed to the distal end of the support member 13 to be supported thereby. In the following descriptions, “axial direction” and “radial directions” designate the axial direction (lengthwise direction) and radial directions of the outer sheath 11, respectively. An end surface of the distal end of the outer sheath 11 is formed as a beveled surface 11a lying in a plane inclined to a plane P (shown by one-dot chain lines in FIG. 11) orthogonal to the axis of the outer sheath 11. The angle of inclination of the beveled surface 11a is approximately 30 degrees. The outer and inner edges of the beveled surface 11a are deburred to ensure a smooth end shape.

The sheet supporting element 23 is a flexible and resilient thin plate member and is provided with a sheet-supporting sheet portion 23a, a connecting portion 23b, and a tapered portion 23c provided between the sheet-supporting sheet portion 23a and the connecting portion 23b. The sheet-supporting sheet portion 23a is configured to support the sheet 30. The connecting portion 23b is positioned at the base (fixed end) of the sheet supporting element 23. The tapered portion 23c is shaped so that the width thereof gradually reduces in the direction from the sheet-supporting sheet portion 23a to the connecting portion 23b. The tapered portion 23c has a pair of side edges which is substantially symmetrical with respect to the axis of the outer sheath 11. Each side edge of this pair of side edges is formed as a combination of a convex arc edge (edge having a predetermined radius of curvature) which is continuous with the sheet-supporting sheet portion 23a, and a concave arc edge (edge having a predetermined radius of curvature) which is continuous with the connecting portion 23b. The resiliency of the sheet supporting element 23 keeps the sheet-supporting sheet portion 23a in an unrolled flat shape as shown in FIGS. 10 and 14 when in a free state, i.e., in a state where no external force is exerted on the sheet supporting element 23.

The sheet supporting element 23 and the slide member 12 are connected to each other via a support member 13. As shown in FIGS. 10 through 13, the support member 13 is provided with two screw holes 13a, an insertion groove 13b and a stepped engaging portion 13c in that order from the front end side of the support member 13. The support member 13 is provided on the stepped engaging portion 13c with a disengaging-movement preventive projection 13d. The support member 13 is provided, on a portion thereof in which the two screw holes 13a are formed, with a flat support surface 13e which is substantially parallel to the axis of the outer sheath 11. The connecting portion 23b of the sheet supporting element 23 is provided with two through holes 23d which are positioned to correspond to the two screw holes 13a of the support member 13, respectively, and is further provided at the rear end of the connecting portion 23b with a bent end 23e that is bent into a substantially right angle and engageable in the insertion groove 13b. When the connecting portion 23b is placed onto the flat support surface 13e with the bent end 23e being engaged in the insertion groove 13b, the two through holes 23d are aligned with the two screw holes 13a of the support member 13, respectively. The sheet supporting element 23 and the support member 13 are secured to each other by two set screws 20 which are screwed into the two screw holes 13a through the two through holes 23d, respectively. Alternatively, the sheet supporting element 23 and the support member 13 can be secured to each other by press-fitting two securing pins into the two screw holes 13a through the two through holes 23d, respectively.

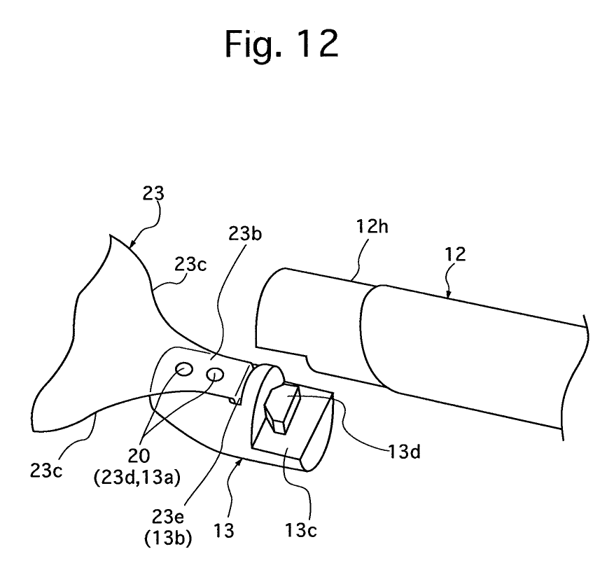

The slide member 12 is provided with a stepped engaging portion 12h which is engaged with the stepped engaging portion 13c of the support member 13, and is provided on the stepped engaging portion 12h with a disengaging-movement preventive recess 12b (shown only in cross section in FIG. 11) in which the disengaging-movement preventive projection 13d of the support member 13 is engaged. As shown in FIG. 12, the disengaging-movement preventive projection 13d that is provided on the support member 13 is a trapezoidal projection, the width of which increases in a direction toward the base end thereof (toward the operating portion) from the distal end of the slide member 12. Due to this shape of the disengaging-movement preventive projection 13d, the slide member 12 and the support member 13 that are separate from each other in the axial direction are prevented from moving relative to each other in directions away from each other by the engagement between the disengaging movement preventive recess 12b and the disengaging-movement preventive projection 13d. On the other hand, the stepped engaging portion 12h and the stepped engaging portion 13c prevent the slide member 12 and the support member 13 from moving relative to each other in directions to approach each other in the axial direction by the engagement of the axially opposed ends of the stepped engaging portion 12h with the stepped engaging portion 13c. In other words, the slide member 12 and the support member 13 are connected to each other so as not to move relative to each other in either forward or rearward directions in the axial direction. This connection can be released by radially moving the slide member 12 and the support member 13 relative to each other.

The junction between the slide member 12 and the support member 13 is positioned to be surrounded by the inner peripheral surface of the outer sheath 11 as shown in FIGS. 10 and 11. Although the support member 13 is prevented from moving relative to the slide member 12 in the axial direction of the outer sheath 11 (horizontal direction with respect to FIG. 11) and can be disconnected only in a radial direction (vertical direction with respect to FIG. 11) of the outer sheath 11 as noted above, the support member 13 and the slide member 12 are prevented from moving relative to each other in this radial direction by the inner peripheral surface of the outer sheath 11 in the state shown in FIG. 11. Namely, the slide member 12 and the support member 13 cannot be disconnected from each other under normal usage, in which the slide member 12 and the rear end of the support member 13 are positioned inside the outer sheath 11.

On the other hand, if the slide member 12 and the support member 13 are made to further project from the outer sheath 11, the slide member 12 and the support member 13 that are otherwise prevented from moving relative to each other by the outer sheath become free from this restriction, which makes it possible to remove the support member 13 from the slide member 12 as shown in FIG. 12.

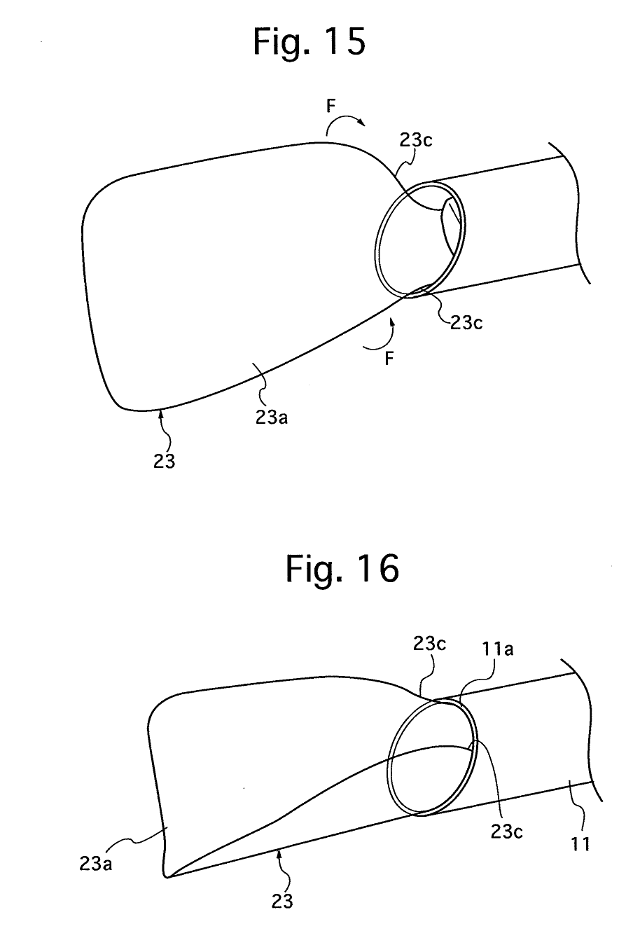

FIG. 14 shows a state where the slide member 12 is pressed into the outer sheath 11 at the forward movement limit of the slide member 12 relative to the outer sheath 11 under normal usage of the therapeutic-substance carrying/administering appliance. In this state, the sheet supporting element 23 projects outwardly from the distal end of the outer sheath 11 and is unrolled so that the sheet-supporting sheet portion 23a is flat due to the shape-sustaining ability (resiliency) of the sheet supporting element 23. The width of the sheet supporting element 23 expanded into a flat shape as shown in FIG. 14 is greater than the inner diameter of the outer sheath 11. Sliding the slide member 12 in a retracting direction indicated by the arrow Si shown in FIG. 14 from the state shown in FIG. 14 causes the tapered portion 23c of the sheet supporting element 23 to come in contact with the beveled surface 11a of the outer sheath 11 as shown in FIG. 15. Thereupon, due to the oblique shapes of the beveled surface 11a and the tapered portion 23c, a component force F (see FIG. 15) urging the sheet supporting element 23 to curl into a tubular shape is produced via the sliding movement of the slide member 12 in the axial direction. The inner surface of the sheet supporting element 23 which is about to become tubular in shape at this time serves as a support surface for supporting the sheet 30.

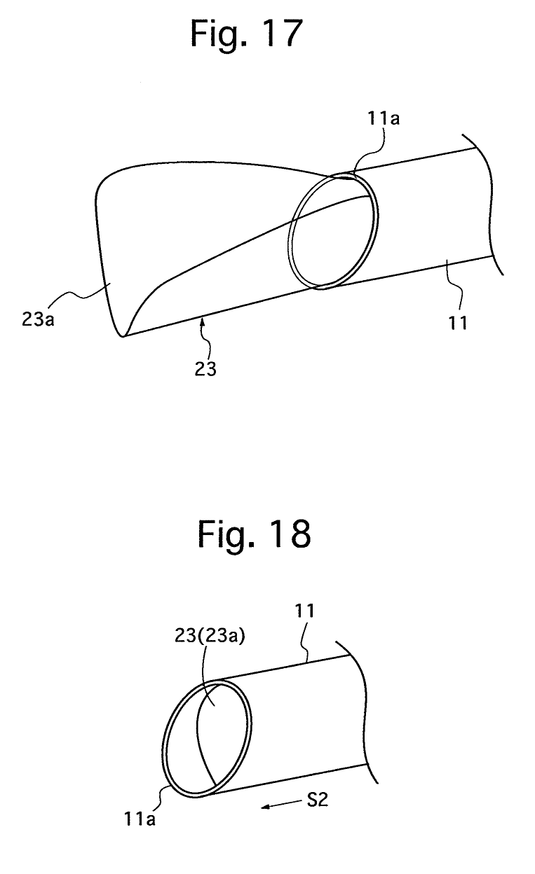

If the slide member 12 continues to be moved in the retracting direction from the state shown in FIG. 15, the sheet supporting element 23 is retracted into the outer sheath 11 to be accommodated therein while being rolled into a tubular shape so as to correspond with the inner peripheral surface of the outer sheath 11 in accordance with the retracting movement of the slide member 12 as shown in FIGS. 16 and 17, in that order. At the stage shown in FIG. 16, the tapered portion 23c of the sheet supporting element 23 is still in contact with the beveled surface 11a of the outer sheath 11, and a component force urging the sheet supporting element 23 to curl into a tubular shape acts on the sheet supporting element 23 in accordance with a retracting movement of the slide member 12 in the axial direction. In a state where the sheet supporting element 23 has been retracted to the stage shown in FIG. 17, the tapered portion 23c has been accommodated in the outer sheath 11; however, at the distal end of the outer sheath 11 (the opening of the outer sheath 11 surrounded by the ring-shaped beveled surface 11a), the opposite side edges of the sheet-supporting sheet portion 23a have been brought closer to each other so as to be adjacent to each other, and the sheet-supporting sheet portion 23a has been almost deformed into a substantially tubular shape. Therefore, even if the slide member 12 is slid in the retracting direction from the state shown in FIG. 17, the sheet supporting element 23 moves toward the front end of the sheet-supporting sheet portion 23a to retract into the outer sheath 11 while being gradually and smoothly rolled into a tubular shape without being snagged on the beveled surface 11a.

FIG. 18 shows a state where the sheet supporting element 23 is fully accommodated in the outer sheath 11. In this state, the sheet supporting element 23 has been deformed into a tubular shape fitted on the inner peripheral surface of the outer sheath 11. The width of the sheet supporting element 23 is determined so that the sheet supporting element 23 does not overlap itself in the tubular accommodated state shown in FIG. 18. Specifically, in the case where the inner diameter of the outer sheath 11 is 9.5 mm, the sheet supporting element 23 can be rolled into a tubular shape fitted on the inner peripheral surface of the outer sheath 11 without overlapping itself if the width of the sheet supporting element 23 is approximately 29 mm.

Contrary to the above described case when the slide member 12 is slid in the retracting direction, sliding the slide member 12 in the projecting direction shown by the arrow S2 shown in FIG. 18 from the accommodated state shown in FIG. 18 causes the sheet supporting element 23 to project outwardly from the distal end of the outer sheath 11 while gradually expanding in directions to become flat by the shape-sustaining ability (resiliency) of the sheet supporting element 23, thus causing the shape of the sheet supporting element 23 to change from the largely deformed state shown in FIG. 18 to the slightly deformed state shown in FIG. 15 via the deformed states shown in FIGS. 17 and 16 in that order. Thereafter, upon the slide member 12 being slid to the maximum projecting position shown in FIG. 14, the tapered portion 23c of the sheet supporting element 23 is disengaged from the beveled surface 11a of the outer sheath 11 to thereby remove restrictions on the shape of the sheet supporting element 23, so that the sheet-supporting plate portion 13a thus having entered a free state expands into a flat shape (original shape).

Accordingly, the sheet 30 is put on the sheet-supporting sheet portion 23a to be held thereby in advance before the outer sheath 11 is inserted into a body, and the sheet-supporting sheet portion 23a can be unrolled at an affected site and the sheet 30 can be transplanted onto the affected site after the outer sheath 11 is inserted into a body.

For instance, the sheet supporting element 23 can be obtained from a resin film with an appropriate thickness which is made of a material such as polypropylene, acrylic resin, polyethyleneterephthalate or polyethylene. Alternatively, the sheet supporting element 23 can also be obtained from a silicon rubber sheet or a thin metal sheet made of a superelastic alloy or a shape-memory alloy. As a matter of convenience of use of the sheet supporting member 23, it is desirable that the sheet supporting element 23 be transparent or translucent. If the sheet supporting element 23 is transparent or translucent, the state of the sheet 30 mounted on the sheet-supporting sheet portion 23a to be supported thereby can be visually checked even from the underside thereof. In addition, during the sheet transplanting operation, one can easily bring the sheet 30 into alignment with an affected site while visually confirming the position of the affected site through the sheet supporting element 23.

As described above, the sheet supporting element 23 is deformed between a flat unrolled shape and a tubular shape in accordance with advancing/retracting movements of the slide member 12 and has the ability (shape-sustaining ability/resiliency) to unroll naturally into a flat shape upon being brought to project out of the outer sheath 11. The orientation of the flat unrolled sheet supporting element 23 is determined by the orientation (rotational angle) of the outer sheath 11, and this orientation can be recognized by visually checking the index mark 20b on the outer sheath 11. On the other hand, the operating portion (the pair of half pinion-holders 16 and the pinion 15) shown in FIGS. 1 through 9 is rotatable about the axial center of the outer sheath 11 (the axial center of the slide member 12), and the slide member 12 can be made to advance and retract regardless of which direction the operating position may be orientated, which is convenient. Moreover, a force to rotate the pinion 15, i.e., the operating portion that includes the pinion 15 and the pair of half pinion-holders 16, is exerted thereon from a direction orthogonal to the outer sheath 11, and accordingly, the outer sheath 11 and the slide member 12 are not carelessly moved in the axial direction during an operation in which the slide member 12 is made to advance and retract in the axial direction. Namely, the positions of the distal ends of the outer sheath 11 and the slide member 12 can be precisely controlled, which makes it possible to enhance the accuracy of the endoscopic operation.

Although the advancing/retracting mechanism for advancing and retracting the sheet supporting element 23 has been discussed above as a desirable embodiment of the slide member advancing/retracting mechanism according to the present invention, the application of the present invention is not limited solely to this particular application.

In the case of using the slide member advancing/retracting mechanism according to the present invention as an advancing/retracting mechanism for advancing and retracting the sheet supporting element 23, a combination of the sheet supporting element 23 and the support member 13, which is detachably attached to the slide member 12, is replaced by new one every time after use. Alternatively, by making the main body of the slide member advancing/retracting mechanism out of a material (e.g., stainless steel), the strength and structure of which being capable of withstanding repetitive use and which is capable of resisting a sterilization treatment, the main body can be reused by sterilizing after use. This reduces the number of disposable parts, thus making it more environmentally efficient and achieving a reduction in the operational cost.

Obvious changes may be made in the specific embodiments of the present invention described herein, such modifications being within the spirit and scope of the invention claimed. It is indicated that all matter contained herein is illustrative and does not limit the scope of the present invention.

What is claimed is:

1. A slide member advancing/retracting mechanism comprising:
an outer sheath;
a slide member installed in said outer sheath to be slidable in an axial direction without rotating relative to said outer sheath; and
an operating portion which can be freely rotated relative to, and about a common axis of said outer sheath and said slide member,
wherein said operating portion can be operated so as to linearly advance/retract said slide member within said outer sheath.

2. The slide member advancing/retracting mechanism according to claim 1, wherein said operating portion comprises:
an operating wheel holder rotatable about said common axis of said outer sheath and said slide member; and
an operating wheel supported by said operating wheel holder to be rotatable on a rotational shaft, an axis of which extends orthogonally to said common axis without intersecting therewith,
wherein said operating wheel and said slide member are engaged with each other so that said slide member linearly moves forward and backward by forward and reverse rotations of said operating wheel, respectively.

3. The slide member advancing/retracting mechanism according to claim 1, wherein said slide member comprises:
an axisymmetrical rack formed integrally with said slide member and having rack teeth in a rotationally-symmetrical shape about said common axis;
wherein said operating portion comprises:
a pinion holder supported by said outer sheath to be rotatable about said common axis; and
a pinion which is supported by said pinion holder to be freely and manually rotatable, and remains in mesh with said axisymmetrical rack regardless of a rotational position of said pinion holder relative to said outer sheath about said common axis.

4. The slide member advancing/retracting mechanism according to claim 1, wherein said slide member comprises a relatively-rotatable slide member which is connected to a rear end of said slide member to be freely rotatable relative to said slide member and to move with said slide member in said axial direction,
wherein said operating portion comprises:
a pinion holder supported by said outer sheath to be rotatable about said common axis; and
a pinion which is supported by said pinion holder to be freely and manually rotatable, and meshes with a rack formed on said relatively-rotatable slide member.

5. The slide member advancing/retracting mechanism according to claim 1, wherein said outer sheath is formed to serve as an endoscopic treatment tool which is inserted into a human body, and
wherein said slide member comprises a support member, provided at a distal end of said slide member, for supporting at least one of a tool and a treatment material which is used for performing a treatment in said human body.

6. The slide member advancing/retracting mechanism according to claim 1, wherein said operating portion is substantially coaxial with the outer sheath.

7. The slide member advancing/retracting mechanism according to claim 2, wherein said rotational shaft is integral with said operating wheel holder.

8. The slide member advancing/retracting mechanism according to claim 2, wherein said operating wheel holder comprises a pair of half holders which holds said pinion therebetween.

9. The slide member advancing/retracting mechanism according to claim 1, wherein said operating portion is fixed at a rear end of said outer sheath.

10. The slide member advancing/retracting mechanism according to claim 1, wherein said slide member advancing/retracting mechanism is incorporated in an endoscopic treatment tool.

11. A slide member advancing/retracting mechanism comprising:
an outer sheath;
a slide member installed in said outer sheath to be slidable in an axial direction thereof without rotating relative to said outer sheath;
an operating portion freely rotatable relative to said outer sheath about said axis of said outer sheath; and
a rack and pinion mechanism installed between said operating portion and said slide member,
wherein manually rotating a pinion of said rack and pinion mechanism causes said slide member to advance and retract in said axial direction via said rack and pinion mechanism.
